(19) United States
(12) Patent Application Publication
(10) Pub. No.: US 2019/0209446 A1
(43) Pub. Date: Jul. 11, 2019

(54) OIL-IN-WATEROIL-IN-WATER TYPE EMULSION HAVING EXCELLENT STABILITY AND COSMETIC COMPOSITION COMPRISING SAME

(71) Applicant: AMOREPACIFIC CORPORATION, Seoul (KR)

(72) Inventors: Ji Yeon HAN, Yongin-si, Gyeonggi-do (KR); Yu Mi SUH, Yongin-si, Gyeonggi-do (KR); Sun Young KIM, Yongin-si, Gyeonggi-do (KR); Ji Sik SHIN, Yongin-si, Gyeonggi-do (KR); Dong Won CHOI, Yongin-si, Gyeonggi-do (KR)

(73) Assignee: AMOREPACIFIC CORPORATION, Seoul (KR)

(21) Appl. No.: 16/328,926

(22) PCT Filed: Aug. 28, 2017
(86) PCT No.: PCT/KR2017/009345

§ 371 Date: Feb. 27, 2019

(30) Foreign Application Priority Data
Aug. 30, 2016 (KR) 10-2016-0110728

(51) Int. Cl. A61K 8/06; A61K 8/19; A61K 8/36; A61K 8/41; A61K 8/44; A61K 8/73; A61K 8/92; A61Q 19/00

(52) U.S. Cl. CPC A61K 8/06; A61K 8/19; A61K 8/36; A61Q 19/00; A61K 8/44; A61K 8/73; A61K 8/92; A61K 8/41

(57) ABSTRACT

An oil-in-water type emulsion and a cosmetic composition having the same to an oil-in-water type emulsion in which an oil-phase part having a C12-C22 fatty acid, an oil and an emulsifier is dispersed in a water-phase part comprising a viscosity increasing agent, a neutralizing agent and water is disclosed. The wherein the oil-in-water type emulsion has a starch-based viscosity increasing agent as the viscosity increasing agent A cosmetic composition having the same is also disclosed. In spite of being an emulsified composition obtained through the neutralization of fatty acids, the oil-in-water type emulsion according to the present invention has excellent stability and is also excellent in view of the feeling of use, skin irritation, and stability on the skin, and thus enables the manufacture and use of various formulations of chemical products.

### TECHNICAL FIELD

The present application claims the benefits of Korean Patent Application No. 10-2016-0110728, filed on Aug. 30, 2016 with the Korean Intellectual Property Office, the disclosure of which is herein incorporated by reference in its entirety.

The present invention relates to an oil-in-water type emulsion in which an emulsion composition obtained through the neutralization of fatty acids not only has an improved stability, but also is excellent in view of the feeling of use, skin irritation, and stability on the skin, and a cosmetic composition comprising the same.

### BACKGROUND ART

Considering complex factors such as sex, age, skin type, and usage, a variety of products with superior quality are being introduced into the cosmetics market. Also, in recent years, as the requirements of customers are becoming more diverse and complicated and the superior quality of the product is a prerequisite, various studies are being conducted to differentiate the product in the appearance, the feeling of use, and the efficacy.

Accordingly, cosmetic products that improve the feeling of use or stability through the diversification of formulations or have various functions such as moisturizing, whitening, and antioxidation in one product are being introduced.

These cosmetics contain various substances such as water-soluble components, oil-soluble components, powders, and active components in the cosmetic composition, are formulated by mixing the substances having different dissolution properties, and have emulsion form through emulsification to absorb more effectively on the skin. In addition, in order to diversify cosmetic formulations and improve the feeling of use, various emulsification techniques are being developed and used.

The emulsion-type cosmetic composition should allow the effect of the oil-soluble components contained therein to be exhibited, should have good feeling of use and should have excellent storage stability from the time the product is opened by the customer until the last use. However, in the case of the emulsion composition, since oil-phase and water-phase components are mixed to form a thermodynamically unstable system, there is an inherent problem that the stability with the passage of time and the change of temperature cannot be permanently sustained. This problem is directly related to the quality of cosmetics and thus various techniques have been developed to stably maintain the oil-soluble components in the cosmetic compositions.

In general, when the cosmetics contain fatty acids as an oil-soluble component, stabilization of the emulsion is achieved through neutralization using basic substances. In this case, the emulsion is effectively stabilized by increasing the neutralization degree of fatty acids through basic substances, but since the pH of the composition rises together, it has been used predominantly in the cleaning products which are applied to the skin and then subsequently rinsed.

Meanwhile, in order to impart moisturizing sustainability and nutritional sensation to skin care products widely used in everyday life, cosmetic products that have increased oil-soluble components by using the relevant stabilization technology have been developed. However, when the neutralization degree of fatty acids is increased in order to ensure the stability of the formulation, the pH is somewhat higher than that of the slightly acidic skin environment, thus causing a problem of irritating the skin. When lowering the neutralization degree in order to solve this problem, it is difficult to ensure consistency and reliability of the quality of the cosmetic product because the free fatty acids that are not neutralized affect the stability of the formulations at high and low temperatures, respectively.

Therefore, there have been various attempts to improve the emulsifying stability, the stability over time, the stability depending on the temperature change, and the feeling of use by varying composition in the emulsion type cosmetic composition.

For example, Korean Patent Publication No. 2015-0108637 discloses an oil-in-water type emulsion cosmetic composition having a feeling of moisture and having water resistance and also having excellent stability by using an emulsification system in which the four raw materials of fatty acids, fatty alcohols and oily waxes are optimally combined.

Also, Korean Patent Publication No. 2014-0145968 discloses a weakly acidic composition for external use on skin which has excellent viscosity and phase stability by using a pH adjusting agent and a polymeric viscosity increasing agent together with a large amount of lipophilic dermatological useful substance.

These patent applications improve the stability of the emulsion composition to some extent, but the effect is not sufficient. Therefore, in the emulsion composition containing fatty acids, it is further required to develop a cosmetic composition having high phase stability and little skin irritation.

### PRIOR ART DOCUMENT

### Patent Document

Korean Patent Publication No. 2015-0108637 (Sep. 30, 2015), STABLE 0/W EMULSION COSMETIC COMPOSITION, and
Korean Patent Publication No. 2014-0145968 (Dec. 24, 2014), WEAKLY ACIDIC COMPOSITION FOR EXTERNAL BASE APPLICATION AND COSMETICS CONTAINING THE SAME.

### DISCLOSURE

### Technical Problem

As a result of various studies to solve the above problems, the inventors of the present invention have confirmed that when the emulsion composition to which the neutralization technology of fatty acids is applied includes a starch-based viscosity increasing agent, it exhibits improved stability and feeling of use and is skin-friendly.

Accordingly, it is an object of the present invention to provide an oil-in-water type emulsion which is safe to the skin because it exhibits excellent emulsifying stability against temperature change while containing fatty acids and has little skin irritation, and a cosmetic composition comprising the same.

### Technical Solution

According to an object of the present invention, the present invention provides an oil-in-water type emulsion in which an oil-phase part comprising a C12-C22 fatty acid, an oil and an emulsifier is dispersed in a water-phase part comprising a viscosity increasing agent, a neutralizing agent and water, wherein the oil-in-water type emulsion comprises a starch-based viscosity increasing agent as the viscosity increasing agent.

The starch-based viscosity increasing agent may comprise at least one selected from the group consisting of starch, carboxymethyl starch, methyl starch, sodium polyacrylate starch and hydroxypropyl starch phosphate.

The oil-in-water type emulsion may comprise a water-phase part comprising 0.01 to 3% by weight of the starch-based viscosity increasing agent, 0.01 to 10% by weight of the neutralizing agent and water as the remainder; and an oil-phase part comprising 0.1 to 20% by weight of the C12-C22 fatty acid, 5 to 30% by weight of the oil and 0.1 to 5% by weight of the emulsifier, based on the total weight of 100% by weight of the oil-in-water type emulsion.

The oil-in-water type emulsion may have a pH of 5 to 8.

In addition, the present invention provides a cosmetic composition comprising the oil-in-water type emulsion.

### Advantageous Effects

The oil-in-water type emulsion according to the present invention can be widely applied to cosmetic products of various formulations because it exhibits improved stability by including a starch-based viscosity increasing agent in the water-phase part, and thus overcoming the limitations of the stability of the emulsion composition obtained by the neutralization of fatty acids, as well as the pH of the composition is hypoallergenic, ranging from slightly acidic to neutral.

### DESCRIPTION OF DRAWINGS

FIG. 1 is a graph showing viscosity changes of the oil-in-water type emulsions according to Example 1 and Comparative Example 1 of the present invention.

FIG. 2 is a photograph showing the result of measurement of the polarizing microscope for the oil-in-water type emulsion according to Example 1 of the present invention immediately after the preparation (a) and after 3 weeks (b).

FIG. 3 is a photograph showing the result of measurement of the polarizing microscope for the oil-in-water type emulsion according to Comparative Example 1 immediately after the preparation (a) and after 3 weeks (b).

### BEST MODE

Hereinafter, the present invention will be described in more detail.

The present invention provides an oil-in-water type emulsion which is excellent in emulsifying stability and usability and at the same time has no skin irritation.

When fatty acids are contained in the cosmetic composition of the emulsion form, the fatty acids are neutralized using a base to form fatty acid salts, thereby being dispersed in the external phase of the dispersion medium in a stable state.

At this time, when the degree of neutralization of the fatty acids is increased in order to improve the emulsifying stability, the pH of the composition is also increased proportionally. Since high pH values act as an irritative factor to the skin, this emulsification technique is mainly applied to a wash-off type cosmetic composition for facial cleansing, such as a make-up remover and a detergent, in which the product stays on the skin in a short period of time.

Recently, in order to provide high moisturizing effect and rich nutritional sensation, as the content of oil-soluble ingredients in skin-care products is increased, corresponding emulsification techniques are being used. In the case of skin care products, since it is a leave-on type that stays in the skin for a long period of time, the composition should be slightly acidic or neutral and thus the degree of neutralization of the fatty acids should be lowered. However, in this case, non-neutralized fatty acids are located at the interface and the water-phase at high temperature, while increasing the viscosity and lowering the fluidity, thereby forming cream-like forms and causing a problem with regard to the high temperature stability with the passage of time. In addition, the crystallization of the non-neutralized fatty acids is accelerated at low temperatures, thereby adversely affecting the appearance and stability.

Accordingly, the present invention provides an oil-in-water type emulsion composition which improves the stability depending on changes in temperature and time, has less skin irritation and exhibits excellent feeling of use, by containing a starch-based viscosity increasing agent in the emulsion composition containing fatty acids.

Specifically, the present invention relates to an oil-in-water type emulsion in which an oil-phase part comprising a C12-C22 fatty acid, an oil and an emulsifier is dispersed into a water-phase part comprising a viscosity increasing agent, a neutralizing agent and water, wherein the oil-in-water type emulsion comprises a starch-based viscosity increasing agent as the viscosity increasing agent.

The water-phase part of the oil-in-water type emulsion according to the present invention provides moisturizing sensation and fresh feeling of use, wherein the emulsified particles including an oil-phase part described later are discontinuously dispersed. The water-phase part comprises the starch-based viscosity increasing agent, the neutralizing agent and water.

The starch-based viscosity increasing agent gives the composition a proper viscosity to increase the feeling of use, and can obtain a better viscosity increasing effect and a moisturizing effect because it has a molecular structure with many branches.

The starch-based viscosity increasing agent may be any of a variety of plant-derived polysaccharides having a glucose repeat unit which is bonded by an alpha acetal bond to form an amylose and/or amylopectin chain. The starch-based viscosity increasing agent may be one obtained from plants such as corn, rice, tapioca, potato, etc., or modified by chemical cross-linking bond.

The starch-based viscosity increasing agent may include at least one selected from the group consisting of starch, carboxymethyl starch, methyl starch, sodium polyacrylate starch and hydroxypropyl starch phosphate. Preferably, the starch-based viscosity increasing agent may be at least one selected from the group consisting of starch, carboxymethyl starch and hydroxypropyl starch phosphate.

The starch-based viscosity increasing agent may be contained in an amount of from 0.01 to 3% by weight, preferably from 0.1 to 2% by weight based on the total weight of 100% by weight of the oil-in-water type emulsion. When the content of the starch-based viscosity increasing agent is less than the above range, the effect of improving the viscosity of the composition cannot be obtained. On the contrary, when the content exceeds the above range, in case of applying to the skin, the problem of degradation of the feeling of use, such as stiffness and stickiness, can be caused.

In particular, the oil-in-water type emulsion according to the present invention includes the starch-based viscosity increasing agent, and thus can stabilize the oil-soluble components contained in an oil-phase part. That is, by including the starch-based viscosity increasing agent, the formulation stability is improved by lowering the fluidity of the oil-soluble components in the composition and minimizing the rheological variance. Accordingly, the various oil-soluble components having the effects of wrinkle improvement, whitening, anti-inflammation, ultraviolet ray shielding and the like can be stably dispersed in the formulation and can be quickly and easily absorbed into skin.

The neutralizing agent serves to enhance the interfacial stability by neutralizing the fatty acids of the oil-phase part described later. The neutralizing agent is a basic substance and can be any substance capable of forming fatty acid salts by reacting with fatty acids.

The neutralizing agent may be a hydroxide, a carbonate, a phosphate, an ammonia or an amine of an alkali metal or an alkaline earth metal, or a basic amino acid. For example, the neutralizing agent may be at least one selected from the group consisting of potassium hydroxide, sodium hydroxide, triethanolamine, methylglucamine, arginine, glycine, alanine, valine, leucine, isoleucine, serine, threonine, aspartic acid, glutamic acid, lysine, ornithine, cysteine, methionine, histidine, phenylalanine, tyrosine and tryptophan. Preferably, the neutralizing agent may be at least one selected from the group consisting of potassium hydroxide, sodium hydroxide, triethanolamine, methylglucamine and arginine.

The neutralizing agent may be contained in an amount of from 0.01 to 10% by weight, preferably from 0.1 to 5% by weight based on the total weight of 100% by weight of the oil-in-water type emulsion. When the content of the neutralizing agent is less than the above range, the fatty acids cannot be sufficiently neutralized. On the contrary, when the content exceeds the above range, it is difficult to use as a cosmetic material because it has excessively high basicity and is slippery.

In the present invention, the following fatty acids and the above neutralizing agent may be contained in a weight ratio of 1:0.1 to 0.5, preferably 1:0.2 to 0.4. When the weight ratio is out of the above-mentioned range, there arises a problem that the viscosity of the composition is changed or precipitation is occurred and thus the stability is lowered.

The water may include distilled water or purified water, and may be used as the remainder based on the total weight of 100% by weight of the oil-in-water type emulsion.

The water-phase part may further include, in addition to the above-mentioned components, a humectant, a viscosity increasing agent, and the like.

The humectant may include propylene glycol, 1,3-butylene glycol, sorbitol, polyglycerin, polyethylene glycol, 1,2-pentanediol, isoprene glycol, erythritol, xylitol, maltitol, glycerin, amino acid, sodium lactate, sodium pyrrolidonecarboxylate, xyloglucan, quince seed, carrageenan, pectin, mannan, cadran, galactan, delmantan sulfate, glycogen, keratan sulfate, chondroitin, mucoitin sulfate, kerato sulfate, locust bean gum, succinoglucan, calonym acid, hyaluronic acid, heparan sulfate, sodium hyaluronate, collagen, mucopolysaccharide, chondroitin sulfate, and the like.

The viscosity increasing agent is an agent for increasing the feeling of use by giving an appropriate viscosity at the time of forming the formulation, and is not particularly limited as long as it is commonly used in the technical field. For example, the viscosity increasing agent may be at least one selected from the group consisting of gelatin, gellan gum, guar gum, xanthan gum, locust bean gum, alginic acid, arabic gum, carrageenan, agar, pectin, rheogic, cellulose, carboxymethylcellulose sodium, hydroxymethyl cellulose, hydroxypropyl methylcellulose, methyl ethyl cellulose, carbomer, acrylate/C10-30 alkyl acrylate cross-polymer, sodium polyacrylate, acrylate/acrylamide copolymer, polyacrylamide, acrylate/beheneth-25 methacrylate copolymer, acrylate/C12-22 alkyl methacrylate copolymer, acrylate/stearath-20 methacrylate cross-polymer, acrylate/stearyl methacrylate copolymer, acrylate/vinyl isodecanoate cross-polymer, acrylate/vinyl neodecanoate cross-polymer and inorganic water-soluble polymer. Preferably, the viscosity increasing agent may include an ionic viscosity increasing agent as a viscosity increasing agent having excellent fluidity and stability to a salt.

The oil-phase part of the oil-in-water type emulsion according to the present invention provides a moisturizing effect and a rich nutritional sensation, and includes the C12-C22 fatty acid, the oil and the emulsifier.

The C12-C22 fatty acid serves to provide a rich nutrition sensation and feeling of use, which can be obtained from formulations such as creams and lotions, and enhance the moisturizing sustainability. The C12-C22 fatty acid can be obtained by hydrolyzing fats with an organic compound having one carboxy group at the end of the hydrocarbon chain. In the present invention, the fatty acid may be at least one selected from the saturated fatty acids of which all carbon atoms in the molecule are saturated and thus represented by a single bond, or the unsaturated fatty acids containing double bond or triple bond in the chain.

The C12-C22 fatty acid is not particularly limited as long as it is generally used in the field of cosmetics. For example, the C12-C22 fatty acid may be lauric acid, myristic acid, palmitic acid, stearic acid, behenic acid, oleic acid, linoleic acid, γ-Linolenic acid, isostearic acid, 12-hydroxystearic acid or undecylenic acid

The C12-C22 fatty acid may be contained in an amount of from 0.1 to 20% by weight, preferably from 0.5 to 15% by weight based on the total weight of 100% by weight of the oil-in-water type emulsion. When the content of the C12-C22 fatty acid is less than the above range, the desired nutrition sensation, feeling of use, and moisturizing effect cannot be improved. On the contrary, when the content exceeds the above range, the stability is lowered and when applied to the skin, the problem in the feeling of use, such as stickiness and greasy shine, can be caused.

The oil helps to dissolve the oil-soluble components including the C12-C22 fatty acid and firmly form an interfacial layer, and may be an oil commonly used in the art.

The oil may be various oils such as hydrocarbon-based oils, ester-based oils, silicone-based oils, and animal or vegetable oils which are in a liquid state. For example, the oil may be at least one selected from the group consisting of polydecene, hydrogenated polydecene, polybutene, hydrogenated polyisobutene, dicaprylyl carbonate, diisostearylmalate, butylene glycol dicaprylate/dicaprate, cetyl 2-ethyl hexanoate, triethylhexanoin, caprylic/capric triglyceride, dicetearyl dimer dilinoleate, diisostearyl malate, dicetearyl dimer dilinoleate, hexyl laurate, pentaerythrityl tetraethylhexanoate, pentaerythrityl tetraisostearate, octyldodecyl stearoyl stearate, cyclopentasiloxane, cyclohexasiloxane, dimethicone, cyclomethicone, phenyl trimethicone, squalane, tamanu oil, macadamia nut oil, sunflower seed oil, olive oil, evening primrose oil, argan oil, apricot oil, sesame oil, orange oil, rosewood oil, bergamot oil, camellia oil, tea tree oil and jojoba oil.

Preferably, the oil may be at least one selected from the group consisting of polydecene, hydrogenated polydecene, cetyl 2-ethylhexanoate, caprylic/capric triglyceride, pentaerythrityl tetraethylhexanoate cyclopentasiloxane, cyclohexasiloxane, dimethicone, squalane and argan oil.

The oil may be contained in an amount of from 5 to 30% by weight, preferably from 10 to 25% by weight based on the total weight of 100% by weight of the oil-in-water type emulsion. When the content of the oil is less than the above range, it is difficult to sufficiently dissolve the oil-soluble components. On the contrary, when the content of the oil exceeds the above range, it is difficult for the oil-phase components to effectively form emulsified particles, and thus the dispersion and long-term stability of the composition may be deteriorated.

The emulsifier serves to lower the interfacial tension between the water-phase part and the oil-phase part, and is not particularly limited as long as it is commonly used in the formation of the oil-in-water type emulsions in the field of cosmetics.

For example, the emulsifier may include at least one selected from the group consisting of cyclopentasiloxane and PEG/PPG-18/18 dimethicone, glycol stearate, sorbitan sesquioleate, glyceryl oleate, glycol distearate, propylene glycol monostearate, glyceryl stearate, sorbitan stearate, PEG-30 dipolyhydroxystearate, PEG-10 dimethicone, cyclopentasiloxane/PEG.PPG-19.19 dimethicone, sorbitan isostearate, lauryl PEG.PPG-18.18 methicone, cetyl PEG.PPG-10.1 dimethicone, lauryl PEG-9 polydimethylsiloxyethyl dimethicone, PPG-26-Buteth-26 and PEG-40 hydrogenated castor oil, PEG-30 hydrogenated castor oil, CETEARETH-12, PEG-5 glyceryl stearate, PEG-60 glyceryl isostearate, PEG-50 stearate, PEG-100 stearate, Polysorbate 20, Polysorbate 80, Polyglyceryl-10 pentastearate, sodium stearoyl lactylate, methoxy PEG-114/polyepsilon caprolactone, potassium cetyl phosphate, Polysorbate 60, Polyglyceryl-10 stearate, sorbitan olivate, inulin lauryl carbamate, cetearyl olivate/sorbitan olivate, PEG-60 hydrogenated castor oil, Polyglyceryl-3 methylglucose distearate, PEG-5 rapeseed sterol, sucrose polystearate/hydrogenated polyisobutene, C12-20 alkyl glucoside, hydrogenated lecithin, arachidyl glucoside, olive oil PEG-8 esters and sucrose fatty acid ester.

Preferably, the emulsifier is a nonionic emulsifier, and for example may be at least one selected from the group consisting of glyceryl stearate, sorbitan stearate, PEG-5 glyceryl stearate, PEG-60 glyceryl isostearate, PEG-50 stearate, PEG-100 stearate and sorbitan olivate.

The emulsifier may be contained in an amount of from 0.1 to 5% by weight, preferably from 0.5 to 3% by weight based on the total weight of 100% by weight of the oil-in-water type emulsion. When the content of the emulsifier is less than the above range, the emulsion is not formed or the stability of the composition is lowered. On the contrary, when the content exceeds the above range, an emulsifier itself has a severe stickiness and causes severe off-flavor, which may adversely affect the feeling of use.

The oil-phase part of the present invention may further contain various components in addition to the above-mentioned components, and for example, wax, higher alcohol and the like may be optionally used.

The wax may be any conventionally used wax, and for example may be carnauba wax, candelilla wax, beeswax, lanolin, ozokerite, ceresin wax, paraffin wax, microcrystalline wax, petrolatum, murumuru seed butter, mango butter, shea butter, cocoa butter, almond butter, pistachio butter, avocado butter, aloe butter, soy butter, wheat berm butter, cupuacu butter, jojoba butter, grape seed butter, hemp butter and the like.

The higher alcohol is not particularly limited as long as it is generally used in the field of cosmetics. The higher alcohol may be C14 to C22 alcohols, and for example may be cetyl alcohol, stearyl alcohol, behenyl alcohol, myristyl alcohol, cetostearyl alcohol, arachidyl alcohol and the like.

In addition, if necessary, it may further include solvents or additives as known in the art. The solvent is used to dissolve oil-soluble components, and may be methanol, ethanol, propanol, and the like. Also, the additives may include pigments, fragrances, preservatives, water-soluble/oil-soluble physiologically-active ingredients, pH adjusting agents, antioxidants, and the like, and may be appropriately selected by those skilled in the art.

Although the oil-in-water type emulsion containing the above-mentioned components is an emulsion formulation obtained through the neutralization of the fatty acids, it is not only superior in stability and the feeling of use by including the starch-based viscosity increasing agent, but also are skin-friendly and can be formulated as leave-on type cosmetics because the pH of the composition corresponds to a mildly acidic to neutral range of 5 to 8, preferably 5.5 to 7.5.

A method for preparing the oil-in-water type emulsion according to the present invention is not particularly limited, and may be a method for preparing an emulsion of the conventional oil-in-water type.

Specifically, the oil-in-water type emulsion is prepared through steps comprising: 1) preparing a water-phase part containing a starch-based viscosity increasing agent and water; 2) preparing an oil-phase part comprising a C8 to C22 fatty acid, an oil, and an emulsifier; 3) adding the oil-phase part to the water-phase part and mixing them and 4) degassing and cooling it.

The mixing process of steps 1) to 4) may be carried out in an agitator capable of controlling the temperature and stirring by a conventional method, and the stirring and heating are applied as necessary.

In addition, the stirring may be performed using a homo-mixer in an agitator, the stirring speed and time may vary depending on the components used, and for example, the stirring may be performed at a speed of from 5000 to 15000 rpm, preferably from 5000 to 10000 rpm, for 3 to 10 minutes. Thereafter, through cooling at 25 to 30° C., the oil-in-water type emulsion of the present invention may be prepared.

Also, the present invention provides a cosmetic composition comprising the oil-in-water type emulsion.

The content of the oil-in-water type emulsion is from 0.0001 to 99.0% by weight based on the total weight of the cosmetic composition, and the content may be appropriately controlled according to the formulation of the cosmetic composition.

When the oil-in-water type emulsion may be used as a cosmetic composition, it can be formulated by adding an acceptable carrier, medium or base which is acceptable in the field of cosmetics. In addition, the cosmetic composition may include adjuvants commonly used in the field of cosmetics, such as fat substances, organic solvents, solubilizers, thickeners and gelling agents, softeners, antioxidants, suspending agents, stabilizers, foaming agents, fragrances, surfactants, water, ionic or nonionic emulsifiers, fillers, chelating agents, preservatives, vitamins, barriers, wetting agents, essential oils, dyes, pigments, hydrophilic activators, lipophilic activators, lipid vesicles or any other components conventionally used in cosmetics. Also, the components may be introduced in amounts commonly used in the field of cosmetics.

Particularly, the cosmetic composition comprising the oil-in-water type emulsion according to the present invention can be easily prepared as leave-on type cosmetics such as toilet water, lotions, creams, and essences, which remain on the skin for a long period of time, because even though it contains fatty acids in the oil-phase part, it is prepared by blending together the starch-based viscosity increasing agents and thus has excellent stability and the pH ranging from 5 to 8 which is in a slightly acidic to neutral range. For example, the cosmetic composition comprising the oil-in-water type emulsion according to the present invention may be a formulation selected from the group consisting of solutions, suspensions, emulsions, pastes, gels, creams, lotions, ointments, powders, soaps, cleansing, patches, packs, foundations and sprays. More particularly, the cosmetic composition may be formulated as skin care products such as emollient toilet water, nourishing toilet water, lotions, essences, creams, gels, packs, patches, masks, mists and the like, make-up products such as make-up bases, foundations, powders, mascaras, lipsticks and the like, make-up removal products such as cleansing oils, cleansing creams, cleansing lotions, cleansing water, point make-up removers and the like, and cleaning products such as soap, cleansing foam, body wash and the like.

When the formulation in the present invention is an ointment, a paste, a cream or a gel, the carrier component may be animal oils, vegetable oils, waxes, paraffin, starch, tragacanth, cellulose derivatives, polyethylene glycol, silicone, bentonite, silica, talc or zinc oxide.

When the formulation in the present invention is a powder or a spray, the carrier component may be lactose, talc, silica, aluminum hydroxide, calcium silicate or polyamide powder, and particularly, when the formulation in the present invention is a spray, propellants such as chlorofluorohydrocarbons, propane/butane or dimethyl ether may be further included.

When the formulation in the present invention is a solution or an emulsion, the carrier component may be a solvent, a solubilizing agent or an emulsifier and may be, for example, water, ethanol, isopropanol, ethyl carbonate, ethyl acetate, glycerin, carbomer, benzyl alcohol, benzyl benzoate, propylene glycol, 1,3-butylglycol oil, glycerol aliphatic ester, caprylic capric triglyceride, hydrogenated polydecene, cetearyl glucoside, sorbitan stearate, polyethylene glycol, cetearyl alcohol and the like.

When the formulation in the present invention is a suspension, the carrier component may be liquid diluents such as water, ethanol or propylene glycol, suspending agents such as ethoxylated isostearyl alcohol, polyoxyethylene sorbitol ester and polyoxyethylene sorbitan ester, and microcrystalline cellulose, aluminum metahydroxide, bentonite, agar or tragacanth.

When the formulation in the present invention is a cleansing, it may be classified into a surfactant-containing cleansing formulation or a surfactant-free cleansing formulation, and it may be applied to the skin and then wiped off or removed or rinsed with water. In the case of the surfactant-containing cleansing, the carrier component may be aliphatic alcohol sulfate, aliphatic alcohol ether sulfate, sulfosuccinic acid monoester, isethionate, imidazolinium derivatives, methyltaurate, sarcosinate, fatty acid amide ether sulfate, alkylamidobetaine, aliphatic alcohols, fatty acid glycerides, fatty acid diethanol amides, vegetable oils, lanolin derivatives, esters of ethoxylated glycerol fatty acids or the like. In addition, the surfactant-containing cleansing formulation may be a cleansing foam, a cleansing water, a cleansing towel, and a cleansing pack, and the surfactant-free cleansing formulation may be a cleansing cream, a cleansing lotion, a cleansing water and a cleansing gel, but is not limited thereto.

When the formulation in the present invention is a soap or a body wash, it may be applied to the skin and then wiped off, removed or washed off with water. As a specific example, the soap may be a liquid soap, a powdered soap, a solid soap, and an oil soap.

Hereinafter, the present invention will be described in more detail with reference to embodiments. It will be apparent to those skilled in the art that these embodiments are only for describing the present invention in more detail and that the scope of the present invention is not limited by these embodiments in accordance with the gist of the present invention.

### Examples and Comparative Examples: Preparation of Oil-in-Water Type Emulsion

Oil-in-water type emulsions were prepared according to the following method using the components shown in Table 1 below.

First, each component of the water-phase part was charged into a stirrer, and completely dissolved by stirring at 70° C. to prepare a water-phase part.

Then, each component of an oil-phase part was charged into a separate mixer and completely dissolved by stirring at 70° C. to prepare an oil-phase part.

Subsequently, the oil-phase part was charged into the water-phase part and stirred at 8500 rpm for 5 minutes at 70° C. using a homo-mixer.

Thereafter, after additionally charging the carbomer, the mixture was stirred at 6000 rpm for 2 minutes with a homo-mixer, and then cooled to 25° C. to prepare an oil-in-water type emulsion.

TABLE 1

| | | | | Compar- | Compar- |
|---|---|---|---|---|---|
| | | | | ative | ative |
| | | | Exam- | Exam- | Exam- |
| Component (% by weight) | | | ple 1 | ple 1 | ple 2 |
| Oil- | Stearic acid | | 2.5 | 2.5 | 2.5 |
| phase | Hydrogenated polydecene | | 10 | 10 | 10 |
| part | Glyceryl stearate | | 1.0 | 1.0 | 1.0 |
| | Cetostearyl alcohol | | 0.5 | 0.5 | 0.5 |
| Water- | Arginine | | 0.5 | 0.5 | 0.5 |
| phase | Purified water | | Remain- | Remain- | Remain- |
| part | | | der | der | der |
| | Viscosity | Hydroxypropyl | 0.5 | — | — |
| | increasing | starch | | | |
| | agent | phosphate | | | |
| | | Carbomer | 0.05 | 0.05 | — |
| | | Hydroxyethyl | — | — | 0.5 |
| | | acrylate/sodium | | | |
| | | acryloyldimeth- | | | |
| | | yltaurate | | | |
| | | copolymer | | | |

### Experimental Example 1: Evaluation of Stability and Safety

1) Fluidity

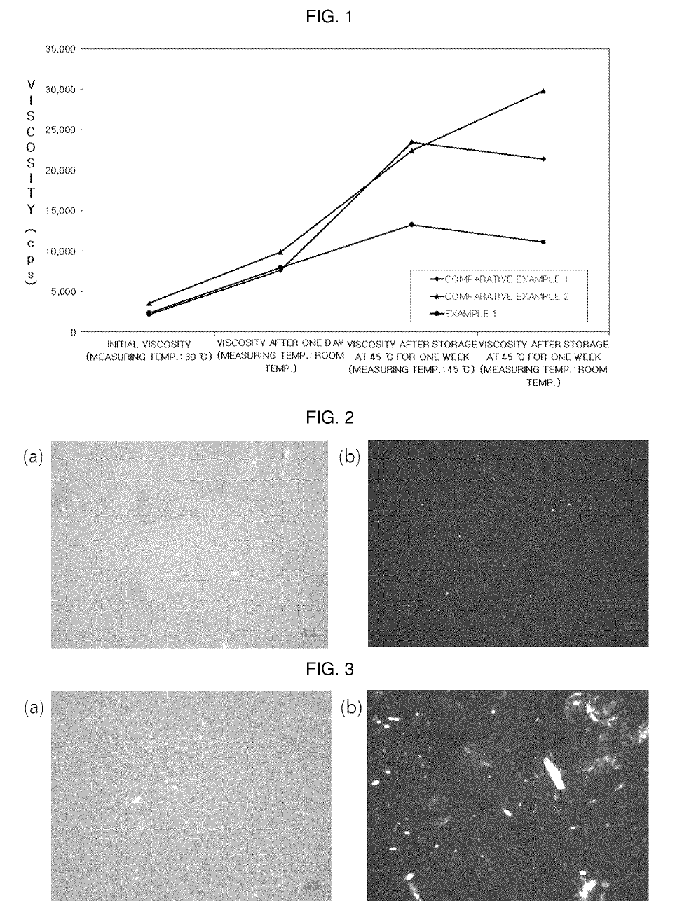

For the oil-in-water type emulsions according to Example 1 and Comparative Example 1, initial viscosity at 30° C. immediately after preparation, viscosity after storage at room temperature for one day, and viscosities at 45° C. and room temperature after storage at 45° C. for one week were measured. The viscosity was measured using a viscometer (DV-2 viscometer, manufactured by Brookfield) under the condition of spindle no. 64 at 30 rpm for 2 minutes, and the results obtained are shown in FIG. 1.

2) Crystallization

The oil-in-water type emulsions prepared in Example 1 and Comparative Example 1 were stored in a thermostatic chamber at −4° C. for 3 weeks while the state of each composition was confirmed by a polarizing microscope (Optiphto-2, manufactured by Nikon). The results obtained at this time are shown in FIGS. 2 and 3.

3) pH

The pH of the undiluted oil-in-water type emulsions prepared in the above Example and Comparative Example was measured using a pH analyzer (METTLER-S20K). The results obtained are shown in Table 2 below.

TABLE 2

| | Example 1 | Comparative Example 1 | Comparative Example 2 |
|---|---|---|---|
| pH | 6.8 | 6.8 | 6.8 |

As can be seen from FIG. 1, the oil-in-water type emulsion of Example 1 according to the present invention has a viscosity of about 66% when stored at high temperature for one week, as compared to the viscosity after one day storage at room temperature. However, it was confirmed that Comparative Example 1 shows about 207% and Comparative Example 2 shows 126%, which is 2 to 3 times higher. Thus, it can be confirmed that the oil-in-water type emulsion according to the present invention has good stability at high temperature.

Also, as shown in FIG. 2, it was confirmed that in the case of the oil-in-water type emulsion of Example 1 according to the present invention, even when stored at a low temperature, the size of emulsified particles is kept constant until three weeks are elapsed, thereby exhibiting excellent stability, whereas the oil-in-water type emulsion prepared in Comparative Example 1 contained uneven emulsified particles due to coalescence between the particles and thus was very low in stability.

From Table 2, it was confirmed that both Example and Comparative Example correspond to a slightly acidic to neutral range. However, as can be seen from FIGS. 1 to 3, it was confirmed that even though the same pH value was exhibited, the emulsion of the Example according to the present invention showed excellent stability with the passage of time and temperature.

### Experimental Example 2: Evaluation of Skin Irritation

Patch test was performed on 20 healthy adult males and females. Specifically, the compositions prepared in Example 1 and Comparative Example 1 were each prepared as an aqueous 1.0% (w/v) solution, and then added in an amount of 0.1_{f}a into a hilltop chamber, and after attaching to the inside of the arms of the 40 subjects and then lapse of 4 hours, the chamber was removed. After removal of the chamber, the degree of skin irritation was visually evaluated by three skin experts. The degree of skin irritation was evaluated based on the criteria shown in Table 3 below, and the results obtained are shown in Table 4 below.

TABLE 3

| | Irritation | Degree of |
|---|---|---|
| Degree of irritation | reaction | irritation |
| Occurrence of erythema, edema and blister | ++ | 3 |
| Occurrence of erythema and edema | + | 2 |
| Occurrence of erythema | +− | 1 |
| No symptoms | − | 0 |

TABLE 4

| | Evaluation result | | | | Average degree |
|---|---|---|---|---|---|
| | ++ | + | +− | − | of irritation |
| Example 1 | 0 | 0 | 0 | 20 | 0 |
| Comparative | 2 | 12 | 4 | 2 | 1.7 |
| Example 1 | | | | | |

As can be seen in Table 4, it was confirmed that the skin irritation index of the composition prepared in Example 1 is generally less than 1, which is a criterion for non-irritation, and thus is suitable for cosmetics that stay on the skin for a long period of time. In contrast, Comparative Example showed a higher skin irritation index than Example.

### Experimental Example 3: Sensory Evaluation

The feeling of use of the skimming and spreading properties and the skin improvement effect of the cosmetic compositions comprising the oil-in-water type emulsions according to Example 1 and Comparative Example 1 were evaluated.

The sensory evaluation was conducted on a group consisting of 30 members. Each composition was used twice daily for 4 weeks, and each item was evaluated according to the following criteria. The total score per item is shown as the average value. The results of the sensory evaluation are shown in Table 3 below.

<Criteria>
X: poor/Δ: normal/o: good/⊚: very good

TABLE 5

| Evaluation item | Example 1 | Comparative Example 1 |
|---|---|---|
| Skimming property | ⊚ | X |
| Spreading property | ⊚ | X |
| Skin improvement effect | ⊚ | Δ |

As can be seen from Table 5, it was confirmed that Example 1 according to the present invention has a remarkably improved feeling of use such as skimming and spreading properties in comparison with Comparative Example 1, and has a better skin improvement effect.

### INDUSTRIAL AVAILABILITY

The oil-in-water type emulsion according to the present invention exhibits improved stability and is excellent in terms of feeling of use, skin irritation and stability on the skin, and thus may be widely applied to cosmetics of various formulations.

1. An oil-in-water type emulsion in which an oil-phase part comprising a C12-C22 fatty acid, an oil and an emulsifier is dispersed into a water-phase part comprising a viscosity increasing agent, a neutralizing agent and water, wherein the viscosity increasing agent comprises a starch-based viscosity increasing agent.

2. The oil-in-water type emulsion according to claim 1, wherein the starch-based viscosity increasing agent comprises at least one selected from the group consisting of starch, carboxymethyl starch, methyl starch, sodium polyacrylate starch and hydroxypropyl starch phosphate.

3. The oil-in-water type emulsion according to claim 1, wherein the oil-in-water type emulsion comprises the water-phase part comprising 0.01 to 3% by weight of the starch-based viscosity increasing agent, 0.01 to 10% by weight of the neutralizing agent and water as the remainder; and the oil-phase part comprising 0.1 to 20% by weight of the C12-C22 fatty acid, 5 to 30% by weight of the oil and 0.1 to 5% by weight of the emulsifier, based on the total weight of 100% by weight of the oil-in-water type emulsion.

4. The oil-in-water type emulsion according to claim 3, wherein the neutralizing agent comprises at least one selected from the group consisting of potassium hydroxide, sodium hydroxide, triethanolamine, methylglucamine, arginine, glycine, alanine, valine, leucine, isoleucine, serine, threonine, aspartic acid, glutamic acid, lysine, ornithine, cysteine, methionine, histidine, phenylalanine, tyrosine and tryptophan.

5. The oil-in-water type emulsion according to claim 1, wherein the oil-in-water type emulsion has a pH of 5 to 8.

6. A cosmetic composition comprising the oil-in-water type emulsion of any one of claim 1.

7. The cosmetic composition according to claim 6, wherein the cosmetic composition is a formulation selected from the group consisting of solutions, suspensions, emulsions, pastes, gels, creams, lotions, ointments, powders, soaps, cleansing, patches, packs, foundations and sprays.
